(19) United States
(12) Patent Application Publication
(10) Pub. No.: US 2021/0358568 A1
(43) Pub. Date: Nov. 18, 2021

(54) NUCLEIC ACID SAMPLE ANALYSIS

(71) Applicant: NOBLIS, INC., Reston, VA (US)

(72) Inventor: Mitchell Yale Holland, Arlington, VA (US)

(21) Appl. No.: 17/444,092

(22) Filed: Jul. 30, 2021

Related U.S. Application Data
(62) Division of application No. 15/881,029, filed on Jan. 26, 2018.
(60) Provisional application No. 62/548,667, filed on Aug. 22, 2017.

(51) Int. Cl. G16B 30/00; G16B 40/00; G16B 20/20

(52) U.S. Cl. CPC G16B 30/00; G16B 20/20; G16B 40/00

(57) ABSTRACT

Systems, methods, apparatus, and technology for nucleic acid sample analysis are provided. For example, a nucleic acid sample may be analyzed to determine whether the sample is contaminated with nucleic acid from multiple individuals. An example method includes receiving sequencing data for a nucleic acid sample, identifying multiple loci in the sequencing data and classify the sequencing data as contaminated by evaluating the multiple loci using a machine learning classifier. In some implementations, the machine learning classifier is trained to provide an output that is an indication of whether the nucleic acid sample includes a particular number of individuals, e.g., one, two, three, four, etc. individuals.

### CROSS REFERENCE TO RELATED APPLICATION

This application is a divisional of, and claims priority to, U.S. application Ser. No. 15/881,029, filed Jan. 26, 2018, which claims the benefit of U.S. Provisional Application No. 62/548,667, filed Aug. 22, 2017, the disclosures of which are incorporated herein by reference in their entirety.

### DISCLOSURE

Sequencing is the process of determining the order of nucleotide bases in a nucleic acid sequence, such as deoxyribonucleic acid (DNA) or ribonucleic acid (RNA). Whole genome sequencing (WGS) is the process of sequencing all of the DNA for an organism (e.g., chromosomal DNA and mitochondrial DNA). DNA sequences are often quite long. For example, WGS data for human DNA (hDNA) from an individual includes approximately six billion nucleotide bases.

Various techniques can be used in sequencing. For example, massively parallel sequencing, which is also referred to as next-generation sequencing or second-generation sequencing, refers to high-throughput techniques for sequencing nucleic acid sequences in many short reads. Multiple copies of a sequence are divided into many smaller sequences that are then sequenced as short reads of, for example, 50-400 nucleotide bases. These short reads are then aligned to determine the order of the nucleotide bases in the original sequence.

### SUMMARY

This disclosure generally relates to nucleic acid sample analysis. For example, a nucleic acid sample may be analyzed to determine whether the sample is contaminated with nucleic acid from multiple individuals. As another example, a nucleic acid sample may be analyzed to infer characteristics of the individual from which the nucleic acid sample originated. Although many of the examples herein are described in terms of DNA, implementations of the systems and techniques described herein can be used with other types of nucleic acid sequences too, such as RNA.

A general aspect is a system comprising a sequencing data receiver and a contaminant determiner. The sequencing data receiver is configured to receive sequencing data for a nucleic acid sample. The contaminant determiner is configured to identify multiple loci in the sequencing data and evaluate the identified loci. The contaminant determiner is also configured to classify the sequencing data as contaminated based on the evaluation of the identified loci.

A general aspect is a method that includes receiving sequencing data for a nucleic acid sample. The method also includes identifying multiple loci in the sequencing data and determining allele read frequencies for the identified loci. The method also includes classifying the sequencing data as contaminated based on the determined allele read frequencies.

A general aspect is a non-transitory computer-readable storage medium comprising instructions stored thereon that, when executed by at least one processor, are configured to cause a computing system to at least receive sequencing data for a nucleic acid sample and identify multiple loci in the sequencing data. The instructions are also configured to cause the computing system to at least determine allele read frequencies for the identified loci and classify the sequencing data as contaminated based on the determined allele read frequencies.

The details of one or more implementations are set forth in the accompanying drawings and the description below. Other features will be apparent from the description and drawings.

### BRIEF DESCRIPTION OF THE DRAWINGS

FIG. 1 is a schematic diagram of an example nucleic acid sample analysis system.

FIG. 2 is an example table of allele read frequency data for SNPs.

FIG. 3 includes a schematic diagram of an implementation of the system of FIG. 1 matching a sample from a crime scene to samples from multiple individuals.

FIG. 4 shows a schematic diagram of an implementation of a sequencing data analyzer of the system of FIG. 1.

FIG. 5 is a flowchart of an example process for analyzing sequencing data from a sample performed by some implementations of the sequencing data analyzer of FIG. 4.

FIG. 6 is a flowchart of an example process for evaluating whether sequencing data is from a sample that is contaminated with nucleic acid sequences from multiple individuals based on a number of variations present at loci performed by some implementations of the sequencing data analyzer of FIG. 4.

FIG. 7 is a flowchart of an example process for evaluating whether sequencing data is from a sample that is contaminated with nucleic acid sequences from multiple individuals based on determined variations that is performed by some implementations of the sequencing data analyzer of FIG. 4.

FIG. 8 is a flowchart of an example process for evaluating whether sequencing data is from a sample that is contaminated with nucleic acid sequences from multiple individuals based on read frequencies of variations at loci that is performed by some implementations of the sequencing data analyzer of FIG. 4.

FIG. 9 shows an example chart of read frequencies and classifications of SNPs.

FIG. 10 is a flowchart of an example process for evaluating whether sequencing data is from a sample that is contaminated with nucleic acid sequences from multiple individuals using a machine learning classifier that is performed by some implementations of the sequencing data analyzer of FIG. 4.

FIG. 11 shows an example chart generated from sequencing data for a sample composed of nucleic acid sequences from a single individual in accordance with at least some implementations.

FIG. 12 shows an example chart generated from sequencing data for a sample composed of nucleic acid sequences from two individuals in accordance with at least some implementations.

FIG. 13 illustrates an example of a computing system that can be used to implement the techniques described here.

Like reference symbols in the various drawings indicate like elements.

### DETAILED DESCRIPTION

The techniques described herein can be used to determine whether a sample, such as WGS data, contains DNA from multiple individuals. In at least some implementations, the techniques described herein do not require any genotype information about the sample.

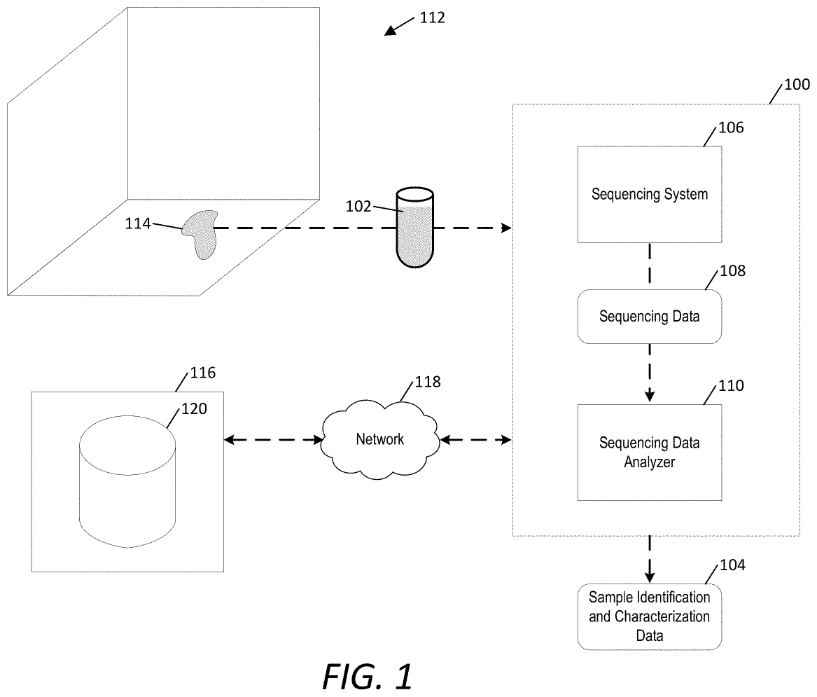

FIG. 1 is a schematic diagram of an example nucleic acid sample analysis system 100. In this example, the system 100 analyzes a nucleic acid sample 102 to generate sample identification and characterization data 104. In some implementations, the sample identification and characterization data 104 includes information to identify an individual associated with the sample 102. The sample identification and characterization data 104 can also include information that indicates whether the sample 102 is from more than one individual. Additionally, the sample identification and characterization data 104 can include information about characteristics of the individual(s) associated with the sample 102.

The sample 102 may be collected in an environment 112 that includes biological material 114 from one or more individuals. For example, the environment 112 may be a crime scene or another environment containing the biological material 114 that may be useful in identifying an individual, such as a suspect. The biological material 114 may be from one or more individuals and may include one or more of teeth, blood, sperm, saliva, bones, hair, skin, urine, and/or feces.

In some implementations, the system 100 includes a sequencing system 106 that generates sequencing data 108 from the sample 102. The system 100 may also include a sequencing data analyzer 110. For example, the sequencing system 106 may generate sequencing data 108 from the sample 102 and the sequencing data analyzer 110 may generate sample identification and characterization data 104 from the sequencing data 108.

The sequencing system 106 may perform massively parallel sequencing on nucleic acid sequences, such as DNA, from the sample 102. The process of performing massively parallel sequencing includes dividing multiple copies of a nucleic acid sequence into shorter sequences of, for example, 50-400 nucleotide bases. The orders of the nucleotides in the shorter sequences are determined to generate sequence reads for the shorter sequences. The smaller sequences from different copies of the nucleic acid sequence will overlap. These overlapping regions are used to align the sequence reads with each other. Additionally or alternatively, one or more reference sequences are used to guide the alignment of the sequence reads from the shorter sequences.

Massively parallel sequencing can be performed more quickly than prior sequencing techniques. However, due to the similarity of DNA from different individuals, the sequence reads for shorter sequences from different individuals can likely be aligned. Thus, if the sample 102 includes nucleic acid sequences from multiple individuals, i.e., the sample 102 is contaminated, it is unlikely that the sequence reads from different individuals will be separated from one another. Instead, a single sequence will be constructed that combines reads from shorter sequences from multiple individuals. This combination of DNA from multiple individuals may interfere with identifying an individual associated with the sample 102.

The sequencing data 108 may include reconstructed sequences that have been determined for some or all of the chromosomes in the sample 102. The sequencing data 108 may also include depth (or coverage) information. The depth information identifies the number of times a particular nucleotide base in a sequence was sequenced. The sequencing data 108 may also include information about allele read frequencies, including single nucleotide polymorphism (SNP) read frequencies. Alleles are alternative forms of a gene and SNPs are changes to a single nucleotide in a sequence and can occur within a gene or in an intron (i.e., a non-coding region) of a nucleic acid sequence. The allele read frequencies and SNP read frequencies can include the number (or percent) of total reads that include a particular allele or a particular nucleotide base at an identified SNP location. Because human cells are diploid (i.e., they have two complete sets of chromosomes, a maternal set and a paternal set), typically hDNA from a single individual will include both homozygous genes in which only a single allele is present (i.e., the maternal chromosome and paternal chromosome both include the same allele of the gene) and heterozygous genes in which two alleles are present (i.e., the maternal chromosome and paternal chromosome include different alleles of the gene). Similarly, an SNP may be present in some reads and not others.

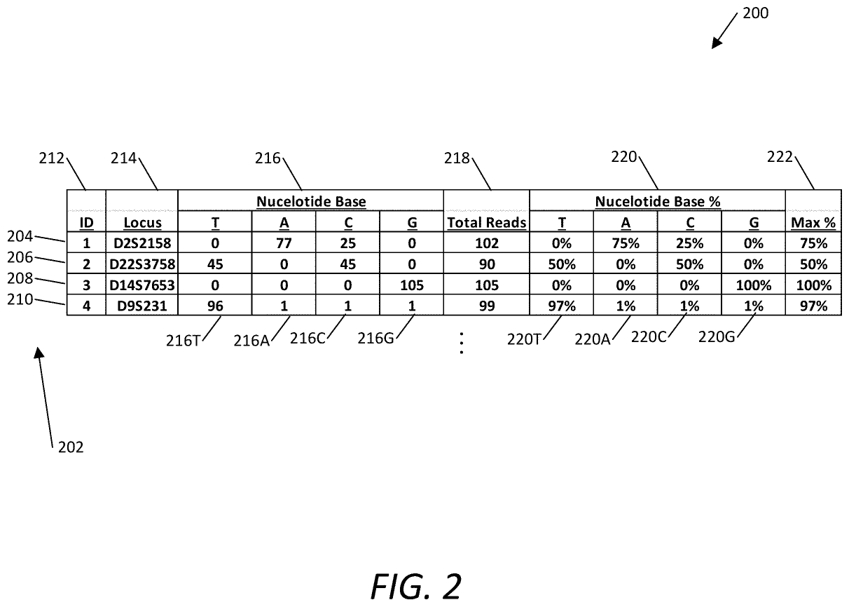

FIG. 2 is an example table 200 of allele read frequency data for SNPs. In this example, the table 200 is storing multiple records 202, including a record 204, a record 206, a record 208, and a record 210. Each of the records 202 represents an example SNP in a nucleic acid sequence from, for example, the sample 102.

For each of the records 202 in the table 200, the table 200 stores various columns containing data. In this example, the table includes an ID column 212, a locus column 214, a nucleotide base column 216, a total reads column 218, a nucleotide base percentage column 220, and a maximum percentage column 222. The ID column 212 may store a unique integer that can be used to identify and/or reference a record in the table 200. The locus column 214 may store information that identifies a locus of the nucleic acid sequence at which the SNP is located. In this example, the locus column stores a textual value for a record that includes a ‘D’ followed by a chromosome number and an ‘S’ followed by a location on the chromosome. In some implementations, other formats are used for storing loci.

The nucleotide base column 216 stores data related to the number of times particular nucleotide bases appeared in reads of the SNP. In this example, the nucleotide base column 216 includes subcolumns 216T, 216A, 216C, and 216G for the nucleotide bases thymine, adenine, cytosine, and guanine respectively. The total reads column 218 includes the total number of reads of the SNP from the sample 102. The records 202 may have different numbers of reads due to, for example, read errors, alignment errors, sample degradation, etc.

The nucleotide base percentage column 220 stores data related to the percentages of each of the nucleotide bases appearing in reads of the SNP. In this example, the nucleotide base percentage column 220 includes subcolumns 220T, 220A, 220C, and 220G for the nucleotide bases thymine, adenine, cytosine, and guanine respectively. For example, the percentage values of the subcolumns 220T, 220A, 220C, and 220G may be determined by dividing the values of the subcolumns 216T, 216A, 216C, and 216G of the nucleotide base column 216 by the value of the total reads column 218 for each of the records 202.

The maximum percentage column 222 stores data corresponding to the maximum percentage value from the nucleotide base percentage column 220. For example, the value of the maximum percentage column 222 may be determined by identifying the maximum percentage value from the subcolumns 220T, 220A, 220C, and 220G for each of the records 202. As described in more detail herein, the maximum percentage values may be used in determining whether a sample is likely to be contaminated.

The table 200 is an example. In some implementations, tables having different, additional, or fewer columns are stored. For example, the nucleotide base percentage column 220 and the maximum percentage column 222 may be calculated by components of the sequencing data analyzer 110 rather than being stored in some implementations. Alternatively, some implementations may store the nucleotide base percentage column 220 and/or the maximum percentage column 222 in a table rather than the nucleotide base column 216 and/or the total reads column 218.

Returning now to FIG. 1, the sequencing data analyzer 110 analyzes the sequencing data 108. For example, the sequencing data analyzer 110 may determine whether the sequencing data 108 indicates that the sample 102 includes nucleic acid sequences from multiple individuals. In some implementations, the sequencing data analyzer 110 determines the number of individuals from which the nucleic acid in the sample 102 came. In some implementations, the sequencing data analyzer 110 characterizes the individual or individuals associated with the sample. For example, the characterization may include identifying a likely continent of origin for the individual and/or likely phenotypic information for the individual (e.g., hair color). In some implementations, the sequencing data analyzer 110 also determines the identity of an individual associated with the sample.

For example, upon determining that the sample 102 is from only one individual, the sequencing data analyzer 110 may communicate with a server 116 over a network 118 to query a database 120. The database 120 may include nucleic acid profiles for multiple individuals. The profiles can include information for multiple loci (i.e., positions) within the nucleic acid sequences in the sample 102. For example, each of the loci may correspond to a designated location within a particular chromosome and may be defined by a chromosome number, an arm, a band, and a sub-band. A locus does not typically map directly to a numeric offset into the sequence. Instead, the portion of the sequence corresponding to the locus is determined after the sequence is mapped to the genes in a chromosome. The profile may include a number corresponding to the length of a short tandem repeat (STR) found at a particular locus. An STR is a short sequence of nucleotide bases that repeats multiple times in a nucleic acid sequence. As another example, the profile may include alleles or single nucleotide polymorphisms (SNPs) at a particular locus.

The network 118 may be a single network or a combination of any type of computer networks, such as a Local Area Network (LAN) or a Wide Area Network (WAN), a WIFI network, a BLUETOOTH network, or other network. In addition, network 118 may be a combination of public (e.g., the Internet) and private networks.

An example of the database 120 is the Combined DNA Index System (CODIS), which is maintained by the U.S. Federal Bureau of Investigation. Querying the database may include identifying a profile from the database that matches the sequence from the sample 102 based on multiple loci from the sample matching a profile.

In some implementations, the sequencing data analyzer 110 may also analyze multiple samples to determine whether any of the samples match each other. For example, a sample may be retrieved from a crime scene and compared to one or more other samples received from respective individuals to determine whether the crime scene sample indicates that one of the individuals was present at the crime scene. In some implementations, the sequencing data for the sample from the crime scene will be analyzed to determine whether the sample includes nucleic acid sequences from a single individual. Responsive to determining that the crime scene sample includes nucleic acid sequences from a single individual, the sequencing data from the sample may be compared to the samples from each of the respective individuals to determine whether any of the individuals match.

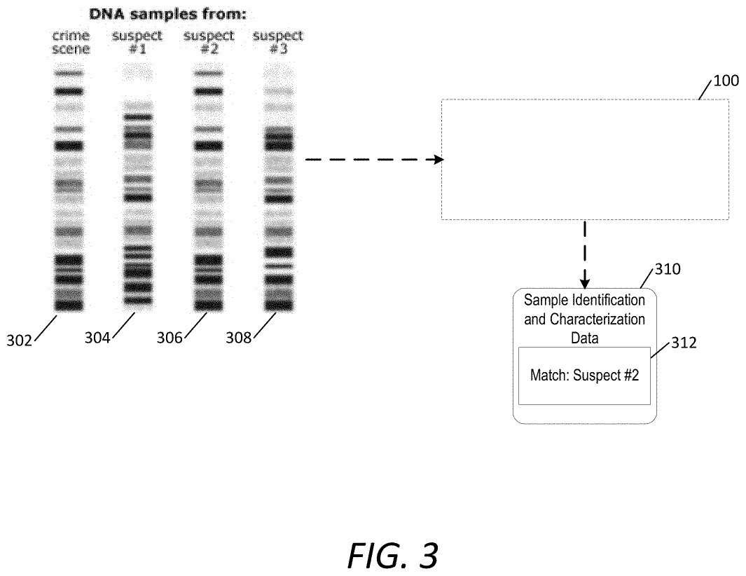

FIG. 3 includes a schematic diagram of the system 100 being used to match a sample 302 from a crime scene to samples 304, 306, and 308 from different individuals. The system 100 generates sample identification and characterization data 310, which is an example of the sample identification and characterization data 104. In this example, the sample identification and characterization data 310 includes an indication 312 that the crime scene sample matches the sample 306 from the individual identified as suspect #2.

Returning now to FIG. 1, in some implementations, the sequencing data analyzer 110 determines whether the sample 102 includes DNA from a single individual or from multiple individuals based on the number of different length values in the sequencing data for STRs at particular loci. The length values represent the number of nucleotide bases in reads of a repetitive sequence of nucleotide bases. For example, if the sequencing data 108 includes more than two different length values for an STR at a particular locus in the sequencing data, it may be determined that the sample 102 includes nucleic acid sequences from more than one individual. Similarly, if more than two different SNPs at the same position or more than two different alleles of the same gene are included in the sequencing data 108, the sequencing data analyzer 110 may determine that the sample 102 includes nucleic acid sequences from more than one individual. In some implementations, the sequencing data analyzer 110 evaluates the number of STR lengths, SNPs, and/or alleles within a nucleic acid sequence and then compares the number to a threshold. In some implementations, if the threshold is exceeded it may be determined that the sample 102 contains nucleic acid sequences from multiple individuals. For example, machine learning techniques and training data may be used to set the threshold or otherwise classify a sample as including nucleic acid sequences from a single individual or from multiple individuals. The machine learning techniques may be applied to labeled training data representing samples from a single individual and samples from multiple individuals. The machine learning technique may train a classifier to classify unlabeled samples as being from a single individual or multiple individuals. In some implementations, the classifier is trained to classify the sample into a class that corresponds to the number of individuals from which the sample comes from.

The classifier may be trained during a training phase. During the training phase, training samples are provided to the classifier along with desired outputs. For example, the desired outputs may include a binary indication whether the sample is contaminated or uncontaminated or a numeric class label that corresponds to the number of individuals from which nucleic acid sequences in the sample come from. The classifier learns the values for various parameters (e.g., weights in layers for a neural network) used in a mapping function that most often result in the desired output when given the inputs.

In some implementations, the machine learning technique includes applying a K-nearest neighbor classification or a similar technique to the sample. The sample may be classified based on the labels of a specific number of the most similar sequences (i.e., the nearest neighbors) in a corpus of training samples. In some implementations, the most similar sequences are identified based on the entire sequences or based on one or more portions of the sequence. For example, the similarity of sequences may be determined by comparing specific loci of the sequences.

The STR, SNPs, and/or alleles may be determined dynamically through analysis of the sequencing data 108, for example by identifying locations in the sequencing data at which different nucleotide bases appear in different reads. In some implementations, the STR, SNPs, and/or alleles may be analyzed at predetermined locations in the sequencing data, such as those identified in the database of short genetic variations (dbSNP) from the National Center for Biotechnology Information (NCBI) or SNP locations identified in the 1000 Genomes Project. Implementations that use predetermined locations, may avoid treating errors in sequence reads or alignment as STRs, SNPs, and/or alleles for purposes of determining whether the sample contains nucleic acid sequences from multiple individuals. In some implementations, machine learning techniques may be used to identify the predetermined set of locations within the nucleic acid sequence.

Some implementations of the sequencing data analyzer 110 determine whether the sample 102 includes DNA from a single individual or multiple individuals based on a panel of SNPs with values that have a positive covariance with one another (i.e., the values of SNPs in the panel are correlated with the values of other SNPs in the panel). For example, the sequencing data analyzer 110 can calculate a probability of a single individual having the SNP values in the sequencing data 108 based on the covariances between the SNP values. If the probability satisfies a condition (e.g., the calculated probability value is less than a predetermined threshold), the sequencing data analyzer 110 determines that the sample 102 contains nucleic acid sequences from multiple individuals. The panel of SNPs may include, for example, between 1000-3000 SNPs.

In some implementations, the sequencing data analyzer 110 determines whether the sample 102 includes DNA from a single individual or multiple individuals based on the read frequencies of alleles and/or SNPs in the sample 102. For example, the alleles and/or SNPs in the sample 102 may be characterized as homozygous, heterozygous, or outliers based on the read frequencies of various alleles or nucleotide values. In some implementations, the alleles and/or SNPs that have a read frequency of greater than 95% are characterized as homozygous, alleles and/or SNPs that have a read frequency of between 40% and 60% are characterized as heterozygous, and the remaining alleles and/or SNPs are characterized as outliers. Based on the percentage of outliers, a probability that the sample 102 includes DNA from multiple individuals is calculated. In some implementations, the percentage of outliers is compared to a threshold value and, if the threshold value is exceeded, it is determined that the sample 102 contains nucleic acid sequences from multiple individuals.

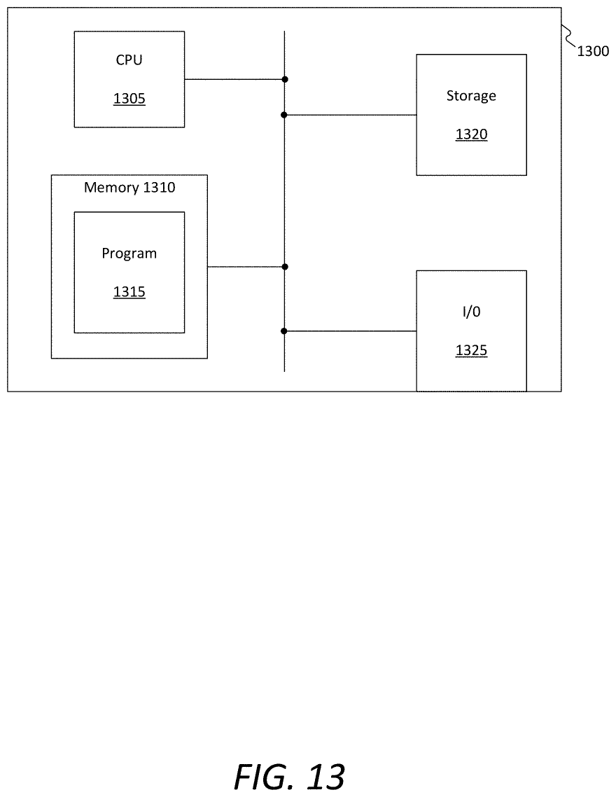

The components illustrated in association with system 100 are merely for illustration, as other components may be included. For example, it will be appreciated that any number of alternative or additional networks, computers, systems, servers, services, mobile devices, or other devices may be included in system 100. Various alternative and additional examples of devices are described in more detail with respect to FIG. 13.

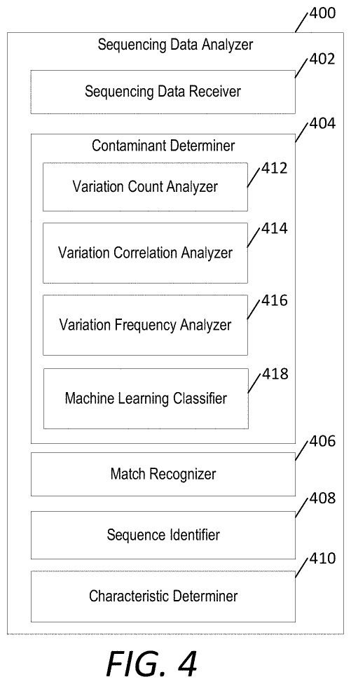

FIG. 4 shows a schematic diagram of an implementation of a sequencing data analyzer 400. The sequencing data analyzer 400 is an example of the sequencing data analyzer 110. The sequencing data analyzer 400 analyzes sequencing data, such as the sequencing data 108. In this example, the sequencing data analyzer 400 includes a sequencing data receiver 402, a contaminant determiner 404, a match recognizer 406, a sequence identifier 408, and a characteristic determiner 410.

The sequencing data receiver 402 receives sequencing data. The sequencing data may correspond to a sample. In some implementations, the sequencing data receiver 402 receives the sequencing data from a local data store. In some implementations, the sequencing data receiver 402 receives the sequencing data from another device via the network 118.

The contaminant determiner 404 determines whether the sequence data is from a sample that is contaminated with nucleic acid sequences from multiple individuals. In this example, the contaminant determiner 404 includes a variation count analyzer 412, a variation correlation analyzer 414, a variation frequency analyzer 416, and/or a machine learning classifier 418. The variation count analyzer 412 analyzes the number of different variations (e.g., alleles, SNPs, STR lengths, etc.) at a particular location in the sequencing data to, for example, determine whether it is likely that the sample is contaminated with nucleic acid sequences from multiple individuals. The variation correlation analyzer 414 analyzes the correlations between multiple of the variations identified in the sequencing data to, for example, determine whether it is likely that the sample is contaminated with nucleic acid sequences from multiple individuals. The variation frequency analyzer 416 analyzes the frequency of different variations at a particular location in the sequencing data to, for example, determine whether it is likely that the sample is contaminated with nucleic acid sequences from multiple individuals.

The machine learning classifier 418 classifies sequencing data from a sample. For example, the machine learning classifier 418 may classify sequencing data based on labeled training data. Some implementations use a classifier trained on labeled training data to classify the sequencing data. In some implementations, the machine learning classifier 418 may use the output of the variation count analyzer 412, the variation correlation analyzer 414, and/or the variation frequency analyzer 416 to classify the sequencing data from a sample.

The training data may include training samples corresponding to sequences or portions of sequences (e.g., specific loci from the sequences that have been identified for use in classification) that are labeled. In some implementations, specific loci are extracted from the training samples prior to training the classifier and then only those specific loci are used to train the classifier. In some implementations, the label may indicate the training sample is either uncontaminated (i.e., includes nucleic sequences from only one individual) or contaminated (i.e., includes nucleic sequences from multiple individuals). In some implementations, the label may indicate the number of individual's samples included in the training sample. The training data, i.e., the training samples, may be generated by sequencing contaminated and uncontaminated samples. In some implementations, the contaminated training data includes synthetic contaminated training data, which may be generated by mixing sequencing data from multiple uncontaminated samples. For example, synthetic contaminated training data may be generated by combining a portion (e.g., half) of the sequencing data from one uncontaminated sample with a portion (e.g., half) of the sequencing data from another uncontaminated sample. In some implementations, the synthetic contaminated training data may be generated from sequencing data from two, three, four, or more individuals. The system may generate labeled training data by associating the number of individual's samples used to generate the training data, and/or by labeling the training data as uncontaminated or contaminated. For example, a training sample generated using samples from three individuals can be labeled with “3” and/or with “contaminated”. Similarly, a training sample generated using one individual's sample can be labeled with “1” and/or with “uncontaminated.” The classifier will be trained to classify a given sample based on the labels. In other words, during training, the classifier will use the labeled training data to correctly predict the label when given its associated training sample. In some implementations, the training process comprises determining weighting parameters for a machine learning classifier such as a logistic regression classifier or a neural network classifier.

The machine learning classifier 418 may be trained by a machine learning module (not shown) of the sequencing data analyzer 400 and/or the machine learning classifier 418 may be trained by and retrieved from another computing device (e.g., such as the server 116) via the network 118. The machine learning classifier 418 may have one of various configurations. For example, the machine learning classifier 418 may include a Naïve Bayesian model, a random tree model, a random forest model, a neural network model, a logistic regression model, or a support vector machine model. The support vector machine model may be trained using, for example, sequential minimal optimization.

In some implementations, the machine learning classifier 418 may classify a sample (e.g., choose a label) for a new sample based on that sample's similarities with characteristics of training samples. In other words, the machine learning classifier 418 may classify an input based on similarity with the learned characteristics of each class (label). In some implementations the machine learning classifier 418 may provide a probability for each class, e.g., a number indicating the likelihood that the sample should be classified in the class.

The match recognizer 406 determines whether a sample from an unknown individual matches one or more other samples. In some implementations, the match recognizer 406 generates a numerical score, such as a probability, representing the strength of a match. In some implementations, the match recognizer 406 uses the classifier predictions of the machine learning classifier 418 to identify matches.

The sequence identifier 408 identifies an individual and/or a profile in a database that matches a sample. In some implementations, the sequence identifier 408 generates a numerical score, such as a probability, representing the certainty of the identification.

The characteristic determiner 410 determines likely characteristics of the individual or individuals associated with a sample. For example, based on the variations identified in the sequencing data, the characteristic determiner 410 may determine a likely heritage and/or phenotype information (e.g., hair color, eye color, skin color, earlobe attachment, etc.) for the individual associated with the sample.

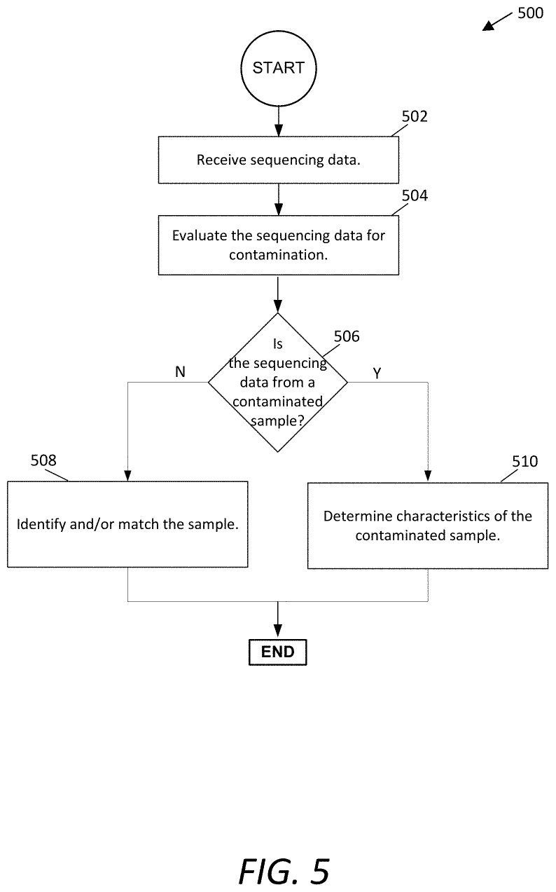

FIG. 5 is a flowchart of an example process 500 for analyzing sequencing data from a sample. The process 500 illustrated in FIG. 5 may be performed at least in part by a sequencing data analyzer, such as the sequencing data analyzer 110 shown in FIG. 1 or the sequencing data analyzer 400 shown in FIG. 4.

At operation 502, sequencing data is received. In some implementations, the sequencing data is received from a sequencing system, such as the sequencing system 106. The sequencing data can also be retrieved from a database or storage device. The sequencing data may be similar to the sequencing data 108, which has been described previously.

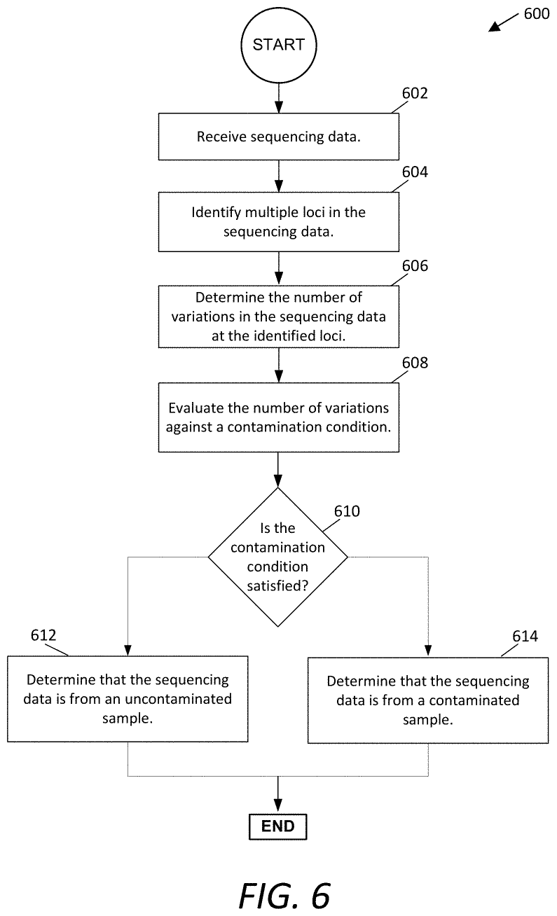

At operation 504, the sequencing data is evaluated for contamination. In some implementations, sequencing data is considered contaminated when the sequencing data was derived from a sample that included nucleic acid sequences from multiple individuals. Various methods for evaluating the sequencing data for contamination are described herein, for example with regard to FIGS. 6-8.

At operation 506, it is determined whether the sequencing data is from a contaminated sample based on the evaluation performed in operation 504. In some implementations, the sequencing data is processed by a machine learning classifier, such as the machine learning classifier 418, to determine whether the sequencing data is contaminated. Example processes of determining whether a sample is contaminated are described with respect to FIGS. 6-8. If it is determined that the sequencing data is not from a contaminated sample, the process 500 continues to operation 508. If instead, it is determined that the sequencing data is from a contaminated sample, the process 500 continues to operation 510.

At operation 508, the sequencing data is used to identify or match the sample. In some implementations, the sample may be used to identify a profile in a database that can then be used to identify an individual. In some implementations, the sequencing data may be compared to other sequencing data from other samples to check for matches. In some implementations, the process 500 then ends. However, in at least some implementations, the process 500 may continue to operation 510 or otherwise determine various characteristics of the sample based on the sequencing data.

At operation 510, the sequencing data is used to determine characteristics of the contaminated sample. Example characteristics include the number of individuals from which the sample came, information about the likely origin of an individual associated with the sample, and phenotypic information about an individual associated with the sample. Such determinations can be accomplished using conventional techniques.

FIG. 6 is a flowchart of an example process 600 for evaluating whether sequencing data is from a sample that is contaminated with nucleic acid sequences from multiple individuals based on a number of variations present at loci. The process 600 illustrated in FIG. 6 is an example process for performing operation 504 from FIG. 5. The process 600 may be performed at least in part by a sequencing data analyzer, such as the sequencing data analyzer 110 shown in FIG. 1.

At operation 602, sequencing data is received. Operation 602 may be similar to previously described operation 502.

At operation 604, multiple loci are identified in the sequencing data. Various number of loci are identified in various implementations. For example, between ten and several thousand loci may be identified. In some implementations, the number of loci identified is determined based on a probabilistic model that predicts the likelihood of multiple individuals matching each of the loci. In some implementations, a machine learning model identifies the loci based on training data. In some implementations, the loci are identified based on identifying positions in the sequencing data in which the sequencing data varies across reads. In some implementations, known SNP or STR locations are used, such as positions identified in the dbSNP from the NCBI or in the 1000 Genomes Project. The loci may also be identified based on using an index that identifies a subset of the sequencing data, identifying loci corresponding to differences from one or more reference sequences (e.g., reference sequences that are used to guide sequence alignment), or selecting loci based on frequency of occurrence of variations in a population (e.g., loci may be selected that have a frequency of occurrence of that meet a condition such as occurring in less than 5%, less than 10%, less than 25%, approximately 50%, or between 45% and 50%). The frequencies of occurrence for specific loci may be determined based on the training data or from other sources. In some implementations, the loci are classified based on percentage occurrence of variations at the loci (e.g., the loci may be classified into classes for each percentile). These classes may then be used to identify specific loci from the sequence data.

At operation 606, the number of variations in the sequencing data at the identified loci is determined. For example, the variations can include different lengths of STRs, different nucleotide bases at an SNP locus, or different alleles of genes. In some implementations, the number of unique lengths of an STR at a particular locus is counted. In some implementations, the number of different nucleotide bases appearing in the sequence data at an SNP locus is counted. Similarly, in some implementations, the number of alleles of a gene at a locus is counted.

At operation 608, the number of variations is evaluated against a contamination condition. An example contamination condition is that a locus has more than two variations, which would be unexpected from a single individual who should have at most two variations at a locus (i.e., one variation from a maternal chromosome and one variation from a paternal chromosome). However, some implementations use other contamination conditions that, for example, may be more robust to sequencing data that includes errors (e.g., due to sequencing or alignment errors). In some implementations, the contamination condition includes a threshold number or threshold percentage of loci that include more than two variations. In some implementations, the contamination condition is based on comparing a weighted sum of the number of variations to a threshold. For example, loci (or variation types) that are more likely to indicate contamination rather than errors in the sequencing data may be given higher weighting values. The threshold and weighting value may be determined by a machine learning model.

At operation 610, it is determined whether the contamination condition is satisfied. If so, the process 600 continues to operation 614, where it is determined that the sequencing data is from a contaminated sample. If not, the process 600 continues to operation 612, where it is determined that the sequencing data is not from a contaminated sample.

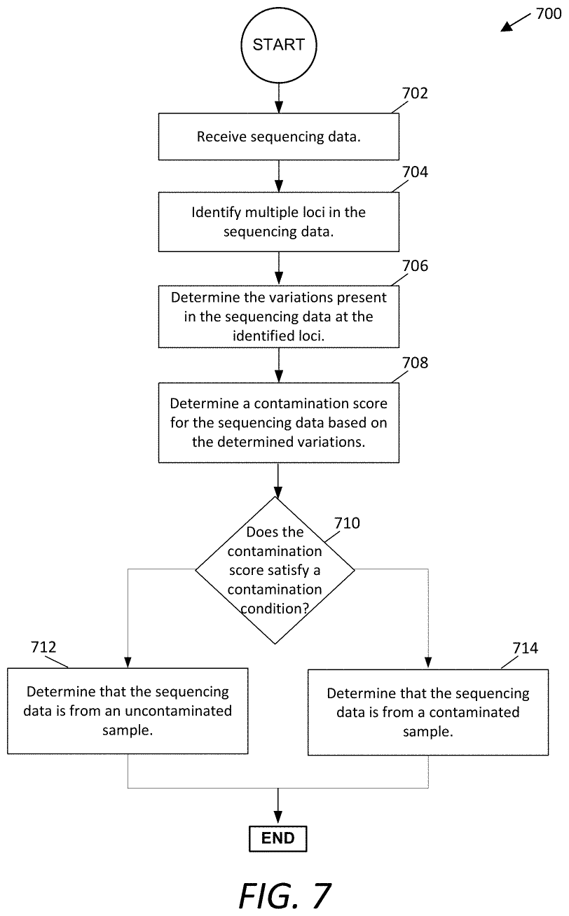

FIG. 7 is a flowchart of an example process 700 for evaluating whether sequencing data is from a sample that is contaminated with nucleic acid sequences from multiple individuals based on determined variations. The process 700 illustrated in FIG. 7 is an example process for performing operation 504 from FIG. 5. The process 700 may be performed at least in part by a sequencing data analyzer, such as the sequencing data analyzer 110 shown in FIG. 1.

At operation 702, sequencing data is received. Operation 702 may be similar to previously described operation 502.

At operation 704, multiple loci are identified in the sequencing data. Operation 704 may be similar to previously described operation 604. As an example, in some implementations, loci associated with SNPs that have a minor allele frequency less than a specific threshold are identified. For example, the threshold may be quite low, such as 1% or even lower. This low threshold ensures that the identified loci are associated with alleles that occur rarely (i.e., the most common allele is observed in most of the population). Minor allele frequency refers to the rate at which the second most common allele is observed in the population. So a minor allele frequency of 1% means the most common allele is observed in 99% of the population. In some implementations, the identified loci are loci that have alleles that are correlated (or inversely correlated) with one another.

At operation 706, the variations present in the sequencing data at the identified loci are determined. As described previously, the variations can include different lengths of STRs, different nucleotide bases at an SNP locus, or different alleles of genes. In some implementations, determining the variations present includes determining whether a minor or major allele is present at a particular SNP locus.

At operation 708, a contamination score is calculated based on the variations that were determined to be present at the identified loci. In some implementations, the contamination score is based on the joint probabilities of at least some of the variations occurring and/or not occurring together. In some implementations, the joint probabilities are determined for identified loci that have a correlative relationship to one another and in which minor alleles were determined to be present. In some implementations, joint probability values are retrieved from a table for each pair of minor alleles determined to be present in the sequencing data.

At operation 710, it is determined whether the contamination score satisfies a contamination condition. An example contamination condition is a probabilistic threshold. For example, in some implementations, the contamination condition is satisfied if the contamination score indicates that the probability of the identified variations occurring in a sample from a single individual is less than 5% (i.e., >95% chance the sample is contaminated).

If the contamination condition is satisfied, the process 700 continues to operation 714, where it is determined that the sequencing data is from a contaminated sample. If not, the process 700 continues to operation 712, where it is determined that the sequencing data is not from a contaminated sample.

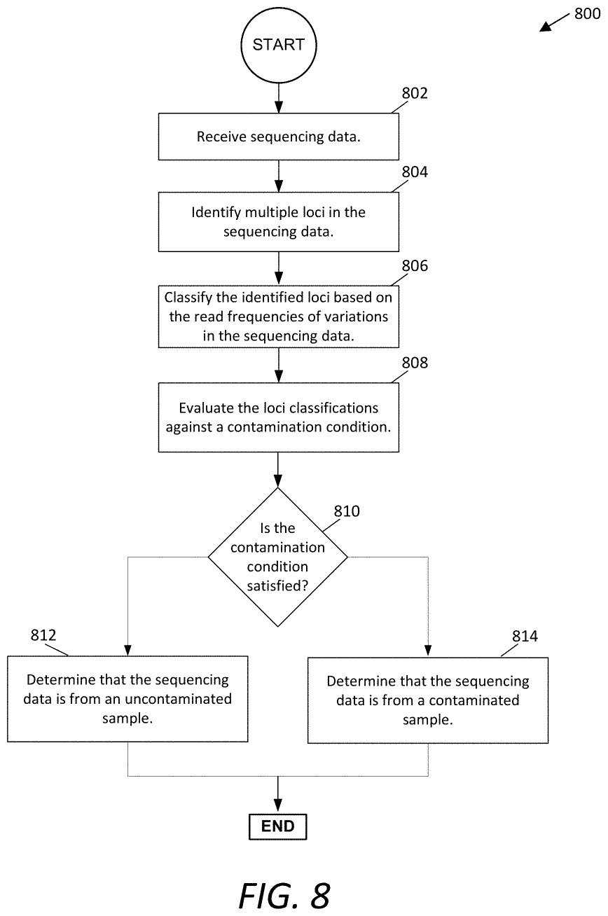

FIG. 8 is a flowchart of an example process 800 for evaluating whether sequencing data is from a sample that is contaminated with nucleic acid sequences from multiple individuals based on loci classification. The process 800 illustrated in FIG. 8 is an example process for performing operation 504 from FIG. 5. The process 800 may be performed at least in part by a sequencing data analyzer, such as the sequencing data analyzer 110 shown in FIG. 1.

At operation 802, sequencing data is received. Operation 802 may be similar to previously described operation 502.

At operation 804, multiple loci are identified in the sequencing data. Operation 804 may be similar to previously described operation 604.

At operation 806, the identified loci are classified based on the read frequencies of the variations present in the sequencing data. As described previously, the variations can include different lengths of STRs, different nucleotide bases at an SNP locus, or different alleles of genes. For example, the identified loci can be classified as homozygous, heterozygous, or outliers based on the read frequencies. In some implementations, the identified loci with a variation having a read frequency of less than 10% or greater than 90% are classified as homozygous; the identified loci with a variation having a read frequency of between 40% and 60% are classified as heterozygous; and the identified loci with a variation having a read frequency of between 5% and 40% or between 60% and 90% are classified as outliers. In some implementations, the identified loci are classified as outliers or non-outliers. For example, the identified loci with a variation having a read frequency of between 10% and 40% or between 60% and 90% are classified as outliers, and all other loci are classified as non-outliers.

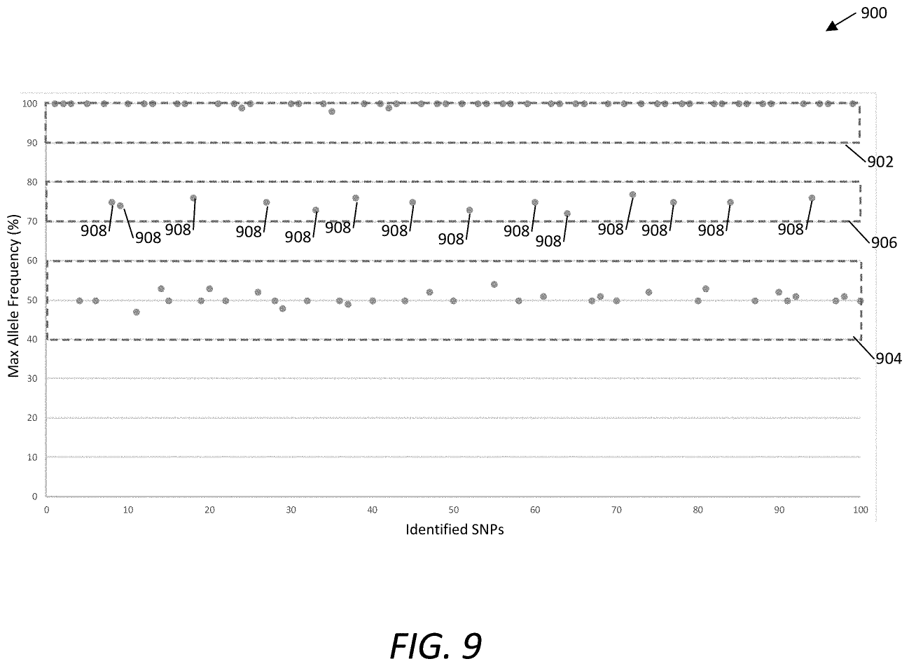

FIG. 9 shows an example chart 900 of read frequencies and classifications of SNPs. The chart 900 includes a homozygous band 902, a heterozygous band 904, and an outlier band 906. The homozygous band 902 identifies regions where the read frequency of the SNP indicates that the SNP is homozygous in the sequencing data. In this example, the homozygous band 902 runs from 90-100%. The heterozygous band 904 identifies regions where the read frequency of the SNP indicates that the SNP is heterozygous in the sequencing data. In this example, the heterozygous band 904 runs from 40-60%. The outlier band 906 identifies regions where the read frequency of the SNP indicates that the SNP is an outlier heterozygous in the sequencing data. In this example, the outlier band 906 runs from 70-80%. Each of the loci classified as outliers is identified at 908 in chart 900. In this example chart 900, the loci are plotted based on the maximum read percentage for the alleles identified at the loci (e.g., the values of the maximum percentage column 222 of FIG. 2). For example, the maximum read percentage may be calculated from the data stored in columns 216 and 218 of FIG. 2 and then classified into the homozygous band 902, heterozygous band 904, or the outlier band 906. In some implementations, if the maximum read percentage for one of the loci does not fit into one of the bands, that locus will be excluded from consideration when determining whether a sample is contaminated.

For example, the records 202 of the table 200 shown in FIG. 2 can be classified using the process 800. For example, the record 204 would be classified as an outlier based on having a maximum percentage of 75% (corresponding to 77 reads of nucleotide base A out of a total of 102 reads); the record 206 would be classified as heterozygous based on having a maximum percentage of 50% (corresponding to 45 reads of nucleotide base T out of a total of 90 reads); the record 208 would be classified as homozygous based on having a maximum read percentage of 100% (corresponding to 105 reads of nucleotide base G out of a total of 105 reads); and the record 210 would also be classified as homozygous based on having a maximum read percentage of 97% (corresponding to 96 reads of nucleotide base T out of a total of 99 reads).

Returning now to FIG. 8, at operation 808, the loci classifications are evaluated against a contamination condition. An example contamination condition is the presence of loci classified as an outlier, which would be unexpected from a single individual who should have nucleic acid sequences that are either homozygous or heterozygous at each of the loci. However, some implementations use other contamination conditions that, for example, may be more robust to sequencing data that includes errors (e.g., due to sequencing or alignment errors). In some implementations, the contamination condition includes a threshold number or threshold percentage of loci that are classified as outliers. In some implementations, the contamination condition may be evaluated based on comparing a weighted sum of the number of loci classified as outliers against a weighted sum of the number of loci classified as homozygous or heterozygous. For example, loci (or variation types) that are more likely to indicate contamination rather than errors in the sequencing data may be given higher weighting values. The threshold and weighting value may be determined by a machine learning model. In some implementations, the evaluation of the contamination condition may be based on the read frequencies of the loci classified as outliers. For example, a read frequency of 75% may be given more weight in evaluating contamination than a read frequency of 6% in some implementations.

At operation 810, it is determined whether the contamination condition is satisfied. If so, the process 800 continues to operation 814, where it is determined that the sequencing data is from a contaminated sample. If not, the process 800 continues to operation 812, where it is determined that the sequencing data is not from a contaminated sample.

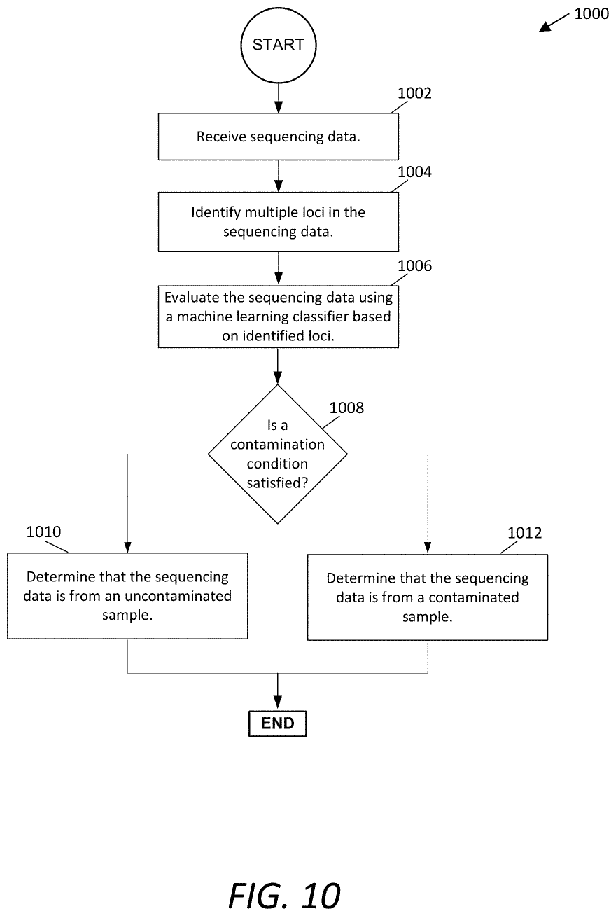

FIG. 10 is a flowchart of an example process 1000 for evaluating whether sequencing data is from a sample that is contaminated with nucleic acid sequences from multiple individuals using a machine learning classifier. The process 1000 illustrated in FIG. 10 is an example process for performing operation 504 from FIG. 5. The process 1000 may be performed at least in part by a sequencing data analyzer, such as the sequencing data analyzer 110 shown in FIG. 1.

At operation 1002, sequencing data is received. Operation 702 may be similar to previously described operation 502.

At operation 1004, multiple loci are identified in the sequencing data. Operation 1004 may be similar to previously described operation 704.

At operation 1006, the sequencing data is classified using a machine learning classifier, such as the machine learning classifier 418, based on the identified loci. For example, the identified loci may be used as input features to the machine learning classifier. The machine learning classifier may then classify the sequencing data based on comparison to learned characteristics of each class, e.g., learned from labeled training data and/or by applying a mapping function to the sequencing data that uses machine learning parameters learned from the labeled training data. The machine learning classifier may be trained in advance using a corpus of labeled training data. In some implementations, the machine learning classifier includes a k-nearest neighbor classifier or similar classifier that uses the identified loci to a specific number of the most similar labeled sequences from the training data. The labels of the training data may then be used to classify the sequencing data. Some implementations use other or additional machine learning classifiers. For example, some implementations use ensemble classifiers that classify the sequencing data based on combining the classification results of multiple machine learning classifiers. In some implementations, the machine learning classifier may generate a binary output indicating whether the sample is contaminated. The machine learning classifier may generate a numeric output, such as a confidence score, that is indicative of the likelihood that the sequencing data is from a contaminated sample. In some implementations, the machine learning classifier may generate a class output that represents the number of individuals in the sample. In some implementations the class output may be associated with a numeric output, such as a confidence or probability score, that is indicative of the likelihood that the sequencing data includes data from that number of individuals.

At operation 1008, it is determined whether the evaluation of the sequencing data satisfies a contamination condition. Depending on the type of machine learning classifier used, different types of contamination condition are used in different implementations. An example contamination condition is a probabilistic threshold. For example, in some implementations, the contamination condition is satisfied if the output from the machine learning classifier indicates that there is a 95% or greater chance that the sample is contaminated and/or includes samples from more than one individual.

If the contamination condition is satisfied, the process 1000 continues to operation 1012, where it is determined that the sequencing data is from a contaminated sample. If not, the process 1000 continues to operation 1010, where it is determined that the sequencing data is not from a contaminated sample. In some implementations, the number of individuals in the contaminated sample may also be determined.

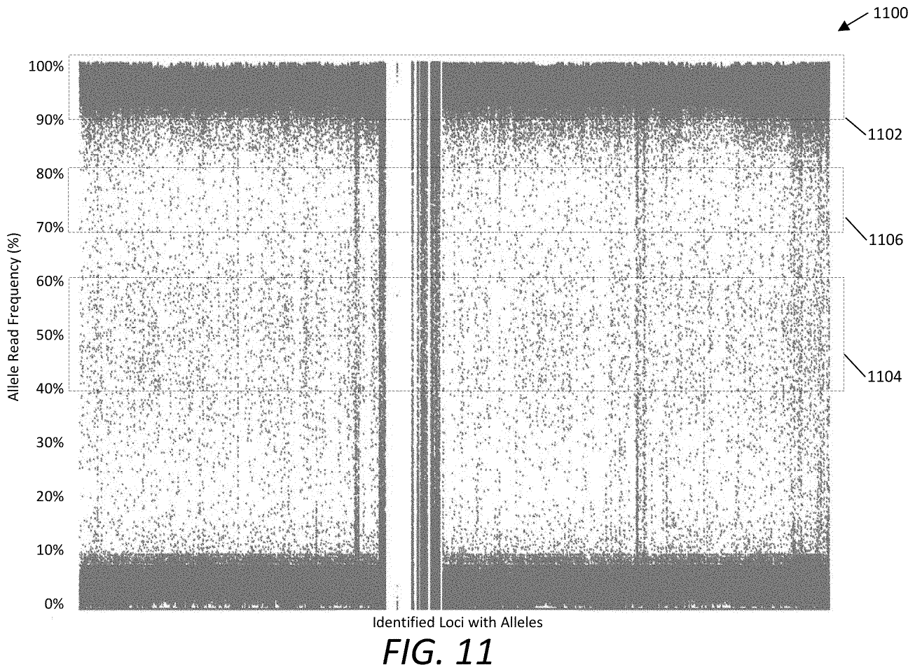

FIG. 11 shows an example chart 1100 generated from sequencing data for a sample composed of nucleic acid sequences from a single individual in accordance with at least some implementations. The chart 1100 includes a homozygous band 1102, a heterozygous band 1104, and an outlier band 1106. The homozygous band 1102, the heterozygous band 1104, and the outlier band 1106 may be similar to the corresponding homozygous band 902, heterozygous band 904, and outlier band 906 of FIG. 9. In this example, loci with alleles identified (e.g., by the sequencing system 106) are arranged along the horizontal axis of the chart 1100. For each of the loci, plot points are added at a vertical location corresponding to the allele read frequency of each of the alleles identified at the locus. As can be seen in the chart 1100, the outlier band 1106 includes some plot points, but the amount of plot points appears uniform relative to the surrounding regions of the chart 1100. Some implementations of the sequencing data analyzer 110 may classify the sequencing data shown in the chart 1100 as not being contaminated due to the amount of plot points in the outlier band 1106 being below a threshold number or percentage or matching a model trained to recognize samples from a single individual.

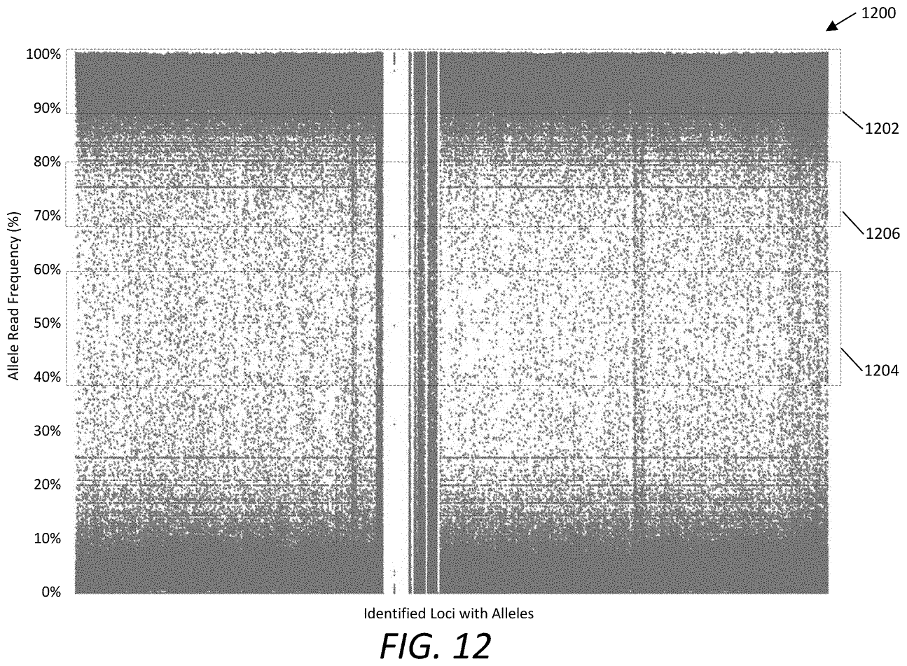

FIG. 12 shows an example chart 1200 generated from sequencing data for a sample composed of nucleic acid sequences from two individuals in accordance with at least some implementations. The chart 1200 includes a homozygous band 1202, a heterozygous band 1204, and an outlier band 1206. The homozygous band 1202, the heterozygous band 1204, and the outlier band 1206 may be similar to the corresponding homozygous band 902, heterozygous band 904, and outlier band 906 of FIG. 9. In this example, loci with alleles identified (e.g., by the sequencing system 106) are arranged along the horizontal axis of the chart 1200. For each of the loci, plot points are added at a vertical location corresponding to the allele read frequency of each of the alleles identified at the locus. As can be seen in the chart 1200, the outlier band 1206 includes a band of plot points at approximately 75%. Some implementations of the sequencing data analyzer 110 may classify the sequencing data shown in the chart 1200 as being contaminated due to the band of plot points at approximately 75% in the outlier band 1206 (e.g., based on meeting a threshold or condition, or fitting a model trained to recognize sample from multiple individuals).

FIG. 13 illustrates an example of a computing system that can be used to implement the techniques described here. The system 1300 is intended to represent various forms of digital computers, such as laptops, desktops, workstations, personal digital assistants, servers, blade servers, mainframes, mobile devices, and other appropriate computers.

The components and arrangement of the system 1300 may be varied. The system 1300 includes a number of components, such as a central processing unit (CPU) 1305, a memory 1310, an input/output (I/O) device(s) 1325, and a nonvolatile storage device 1320. The system 1300 can be implemented in various ways. For example, an integrated platform (such as a workstation, personal computer, laptop, etc.) may comprise the CPU 1305, the memory 1310, the nonvolatile storage 1320, and I/O devices 1325. In such a configuration, components 1305, 1310, 1320, and 1325 may connect through a local bus interface and access a database, such as the database 120, via an external connection. This connection may be implemented through a direct communication link, a local area network (LAN), a wide area network (WAN) and/or other suitable connections. The system 1300 may be standalone or it may be part of a subsystem, which may, in turn, be part of a larger system.

The CPU 1305 may be one or more processing devices, such as a microprocessor. The memory 1310 may be one or more storage devices configured to store information used by CPU 1305 to perform certain functions related to implementations of the present invention. The storage 1320 may be a volatile or non-volatile, magnetic, semiconductor, tape, optical, removable, nonremovable, or other type of storage device or computer-readable medium. In one implementation, the memory 1310 includes one or more programs or subprograms 1315 loaded from storage 1320 or elsewhere that, when executed by the CPU 1305, perform various procedures, operations, or processes consistent with the processes described here. For example, memory 1310 may include programs corresponding to components of the nucleic acid sample analysis system, such as the sequence data analyzer and that execute instructions to perform one or more of the process described in FIGS. 6-8. The memory 1310 may also include other programs that perform other functions and processes, such as programs that provide communication support, Internet access, etc.

Methods, systems, and articles of manufacture described here are not limited to separate programs or computers configured to perform dedicated tasks. For example, the memory 1310 may be configured with a program 1315 that performs several functions when executed by CPU 1305. For example, the memory 1310 may include a single program 1315 that performs the functions of a nucleic acid sample analysis system. Moreover, the CPU 1305 may execute one or more programs located remotely from system 1300. For example, the system 1300 may access one or more remote programs that, when executed, perform functions related to implementations described here.

The memory 1310 may be also be configured with an operating system (not shown) that performs several functions when executed by CPU 1305. The choice of operating system, and even to the use of an operating system, is not critical.

I/O device(s) 1325 may include one or more input/output devices that allow data to be received and/or transmitted by system 1300. For example, the I/O device 1325 may include one or more input devices, such as a keyboard, touch screen, mouse, etc., that enable data to be input from a user, such as sequencing and analysis requests, adjustment of threshold and contamination conditions, etc. Further, the I/O device 1325 may include one or more output devices, such as a display screen, CRT monitor, LCD monitor, plasma display, printer, speaker devices, and the like, that enable data to be output or presented to a user. The I/O device 1325 may also include one or more digital and/or analog communication input/output devices that allow computing system 1300 to communicate with other machines and devices, such as other continuous remote servers that process sample profile queries. The system 1300 may input data from external machines and devices and output data to external machines and devices via I/O device 1325. The configuration and number of input and/or output devices incorporated in the I/O device 1325 are not critical.

The systems and techniques described here can be implemented in a computing system that includes a back-end component (e.g., as a data server), or that includes a middleware component (e.g., an application server), or that includes a front-end component (e.g., a client computer having a graphical user interface or a Web browser through which a user can interact with an implementation of the systems and techniques described here), or any combination of such back-end, middleware, or front end components. The components of the system can be interconnected by any form or medium of digital data communication (e.g., a communication network). Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

A number of implementations have been described. Nevertheless, it will be understood that various modifications may be made without departing from the spirit and scope of the invention.

In addition, the logic flows depicted in the figures do not require the particular order shown, or sequential order, to achieve desirable results. In addition, other steps may be provided, or steps may be eliminated from the described flows, and other components may be added to, or removed from, the described systems. Accordingly, other implementations are within the scope of this disclosure.

What is claimed is:

1. A system comprising:
at least one processor; and
memory storing instructions that, when executed by the at least one processor, cause the system to:
receive sequencing data for a nucleic acid sample,
identify multiple loci in the sequencing data, and
classify the sequencing data as contaminated by evaluating the multiple loci using a machine learning classifier.

2. The system of claim 1, wherein the machine learning classifier is trained using labeled training data comprising sequence data for a plurality of samples and associated labels, wherein a label for an associated sample indicates whether the associated sample is contaminated.

3. The system of claim 1, wherein the machine learning classifier includes multiple classifiers, each classifier of the multiple classifiers being configured to generate a binary output.

4. The system of claim 3, wherein the binary output is an indication of whether the nucleic acid sample includes a particular number of individuals, each classifier of the multiple classifiers indicating a different number of individuals.

5. The system of claim 4, wherein the particular number ranges from one to four.

6. The system of claim 3, wherein the machine learning classifier is an ensemble classifier that combines results of the multiple classifiers.

7. The system of claim 1, wherein the machine learning classifier is trained using labeled training data, the labeled training data including synthetic contaminated training data generated by mixing sequencing data from a known number of uncontaminated samples.

8. The system of claim 7, wherein the known number is one of one, two, three, or four and labels for the synthetic contaminated training data reflect the known number.

9. The system of claim 1, wherein identifying the multiple loci in the sequencing data includes identifying loci within the sequencing data based on predetermined frequencies of occurrence of variations for the loci in a population.

10. The system of claim 1, wherein identifying the multiple loci includes using machine learning techniques.

11. The system of claim 1, wherein the machine learning classifier also produces a confidence score indicative of a likelihood that the multiple loci include data from a predicted number of individuals.

12. A method comprising:
receive sequencing data for a nucleic acid sample;
identify multiple loci in the sequencing data; and
classify the sequencing data as contaminated by evaluating the multiple loci using a machine learning classifier.

13. The method of claim 12, wherein the machine learning classifier includes multiple classifiers, each classifier of the multiple classifiers being configured to generate a binary output.

14. The method of claim 13, wherein the binary output is an indication of whether the nucleic acid sample includes a particular number of individuals, each classifier of the multiple classifiers indicating a different number of individuals.

15. The method of claim 13, wherein the machine learning classifier is an ensemble classifier that combines results of the multiple classifiers.

16. The method of claim 12, wherein the machine learning classifier is trained using labeled training data, the labeled training data including synthetic contaminated training data generated by mixing sequencing data from a known number of uncontaminated samples.

17. The method of claim 16, wherein the known number is one of one, two, three, or four and labels for the synthetic contaminated training data reflect the known number.

18. The method of claim 16, wherein identifying the multiple loci includes using machine learning techniques.

19. The method of claim 12, wherein the machine learning classifier also produces a confidence score indicative of a likelihood that the multiple loci include data from a predicted number of individuals.

20. A non-transitory computer-readable storage medium comprising instructions stored thereon that, when executed by at least one processor, are configured to cause a computing system to at least:
receive sequencing data for a nucleic acid sample;
identify multiple loci in the sequencing data; and
classify the sequencing data as contaminated by evaluating the multiple loci using a machine learning classifier.
